(19) United States
(12) Patent Application Publication
(10) Pub. No.: US 2024/0392357 A1
(43) Pub. Date: Nov. 28, 2024

(54) ONE-POT SINGLE-STRANDED DNA CYCLIZATION AMPLIFICATION AND CRISPR/CAS-MEDIATED NUCLEIC ACID MOLECULE DETECTION METHOD

(71) Applicant: SHANGHAI JIAO TONG UNIVERSITY, Shanghai (CN)

(72) Inventors: Litao YANG, Shanghai (CN); Zaobing ZHU, Shanghai (CN); Yongkun GUO, Shanghai (CN)

(73) Assignee: SHANGHAI JIAO TONG UNIVERSITY, Shanghai (CN)

(21) Appl. No.: 18/800,087

(22) Filed: Aug. 11, 2024

Related U.S. Application Data
(63) Continuation of application No. PCT/CN2022/140596, filed on Dec. 21, 2022.

(30) Foreign Application Priority Data
Jun. 21, 2022 (CN) 202210704969.1

(51) Int. Cl. C12Q 1/6844; C12Q 1/682

(52) U.S. Cl. CPC C12Q 1/6844; C12Q 1/682

(57) ABSTRACT

Disclosed is a one-pot single-stranded DNA cyclization amplification and CRISPR/Cas-mediated nucleic acid molecule detection method. The method comprises: single-stranded DNA probe cyclization, rolling circle amplification, double-probe CRISPR/Cas cleavage identification of single-stranded DNA, and single-tube one-pot detection reaction. According to the present disclosure, in order to mainly solve the problem that cyclization, amplification and cleavage identification cannot be synchronously carried out, an OPERATOR technology for three-step reaction, simultaneous cohesion of cyclization, amplification and cleavage identification, is adopted, and nucleic acid molecules are accurately, sensitively and rapidly detected in an in-vitro single reaction tube system. Compared with a traditional nucleic acid molecule detection method, the method has the following advantages that: the method is suitable for sensitive and rapid detection of single-stranded DNA, double-stranded DNA and RNA molecules. Particularly, when detected, RNA is detected directly by one-pot reaction without reverse transcription, and step-by-step reaction is not needed.

### CROSS-REFERENCE TO RELATED APPLICATIONS

This application is a Continuation of International Application No. PCT/CN2022/140596, filed on Dec. 21, 2022, which claims priority to Chinese Patent Application No. 202210704969.1, filed on Jun. 21, 2022, the entire contents of each of which are hereby incorporated by reference.

### SEQUENCE LISTING

The instant application contains a Sequence Listing which has been submitted electronically in XML format and is hereby incorporated by reference in its entirety. The XML copy, created on Jul. 2, 2024, is named “Sequence Listing-65617-H022US00” and is 10,577 bytes in size.

### TECHNICAL FIELD

The present disclosure relates to the field of biological nucleic acid molecular detection, and in particular, to a one-pot single-stranded DNA cyclization amplification and CRISPR/Cas-mediated nucleic acid molecular detection method.

### BACKGROUND

Deoxyribonucleic acid (DNA) amplification of nuclear detection reactions by quantitative real-time polymerase chain reaction (qPCR) is currently the gold standard for molecular diagnosis. For in vitro detection of ribonucleic acid (RNA), the process typically involves two distinct stages. First, an RNA sample undergoes reverse transcription to form a complementary DNA (cDNA) sample, and then, the cDNA sample is analyzed by the conventional qPCR. Although commercially available reverse transcription-quantitative real-time polymerase chain reaction (RT-qPCR) kits are available, their sensitivities can vary significantly, with single test sensitivities ranging from 45% to 60% (Al-Tawfiq & Memish, 2020). In addition, RT-qPCR detection requires expensive thermal cyclers, experienced operators, and high quality of extracted RNA, and the testing time of RT-qPCR is long (at least 2-4 h from sample processing to result readout), limiting its application in Point of Care Test (POCT).

In recent years, with the development of nucleic acid isothermal amplification technology, its role in nucleic acid molecular diagnosis has become more and more prominent, isothermal amplification technology mainly utilizes the displacement of amplifying enzymes in constant temperature and polymerase properties, which can realize the efficient amplification of specific targets under the spontaneous action of primers. A variety of nucleic acid isothermal amplification methods have been derived, such as the loop-mediated isothermal amplification (LAMP) technique, recombinase polymerase amplification (RPA) technique, rolling circle amplification (RCA) method, and nucleic acid sequence-based amplification (NASBA) technique.

However, the above several isothermal amplification techniques all have their own shortcomings. For example, the LAMP technique requires several primers (4-6), which is prone to aerosol contamination in on-site detection, resulting in false positive results, and amplification of mutation sites is almost unavoidable. The enzyme component of the RPA method is more complex and does not allow for the detection of mutation sites, while a reverse transcription step is still required for RNA samples. The RCA method lack validity and specificity for the detection of products of amplified single-stranded DNA.

Clustered Regularly Interspaced Short Palindromic Repeat (CRISPR) systems are a type of natural “immune system” widely found in bacteria and archaea. As an adaptive immune mechanism, the CRISPR system can recognize exogenous genetic material and integrate it into the CRISPR sequences of the organism's own genome. When the exogenous genetic material invades again, a Cas nuclease precisely cleaves the exogenous nucleic acids. The Cas nuclease is an important CRISPR-associated protein, and several CRISPR-associated proteins have been identified, such as Cas9, Cas12, Cas13, Cas14, etc., among which the latter three (Cas12, Cas13, Cas14) have been widely used in the field of nucleic acid diagnostics because of their cis-and trans-cleavage activities. In 2019, Wang Jin's group developed HOLMES (S. Y. Li et al., 2018), HOLMESv2 (L. Li et al., 2019) detection systems using Cas12 combined with LAMP technique, which can reach a sensitivity of about 10-8 nM. However, the HOLMES method does not allow for direct detection of RNA samples, while integrated, single-tube detection is not possible due to temperature differences between amplification and detection.

Therefore, there is a need to provide an integrated, sensitive, specific, and rapid detection method for the detection of nucleic acid molecules.

### SUMMARY

One or more embodiments of the present disclosure provide a one-pot single-stranded DNA cyclization amplification and CRISPR/Cas-mediated nucleic acid molecule detection method, comprising:
step (1): extracting a nucleic acid sample from a test sample;
step (2): preparing a reaction system mixture, the reaction system mixture comprising: a single-stranded DNA probe, a dual fluorescently labelled single-stranded DNA probe, an oligonucleotide primer, a DNA ligase or variants thereof, a strand displacing DNA polymerase or variants thereof, a guide RNA or derivatives thereof, a CRISPR-associated Cas protein or variants thereof, an OPERATOR reaction buffer; wherein the guide RNA or derivatives thereof comprises a sequence identical to a sequence of the nucleic acid molecule to be detected, the single-stranded DNA probe is specifically complementary to a strand of a nucleic acid molecule to be detected in the nucleic acid sample, and a backbone sequence of the single-stranded DNA probe includes a PAM site sequence and a random ligation sequence in addition to a sequence of a complementary target portion or a derivative thereof;
step (3): adding the nucleic acid sample to the reaction system mixture for a constant temperature reaction; and
step (4): producing a detectable fluorescent signal after the dual fluorescently labelled single-stranded DNA probe is cleaved, reading and recording the fluorescent signal, and obtaining a nucleic acid detection result.

In some embodiments, in step (1), the nucleic acid molecule to be detected in the nucleic acid sample comprises one or more of single-stranded DNA, double-stranded DNA, or single-stranded RNA.

In some embodiments, in step (2), the oligonucleotide primer is a base-modified random primer or a primer consistent with the sequence of the nucleic acid molecule to be detected.

In some embodiments, in step (2), the sequence of the dual fluorescently labelled single-stranded DNA probe is complementary to the sequence of the nucleic acid molecule to be detected, and the 5′ end and the 3′ end of the dual fluorescently labelled single-stranded DNA probe are labelled with fluorescent moieties, respectively; and the fluorescent moiety at the 5′ end comprises one of FAM, HEX, VIC, Cy5, Cy3, ROX, FITC, and Joe, and the fluorescence quenching moiety labelled at the 3′ end comprises one of TAMRA, BHQ1, MGB, and BHQ2.

In some embodiments, in step (2), the DNA ligase is a ligase that joins single-stranded DNA nicks of a double-stranded DNA molecule or an RNA and DNA hybrid double strand. The DNA ligase comprises one of T4 DNA ligase, E. coli DNA ligase, SplintR ligase, and HiFi Taq DNA ligase. The strand displacing DNA polymerase includes one of Phi29, Klenow, and Vent.

In some embodiments, in step (2), the CRISPR-associated Cas protein is a CRISPR-Cas nuclease having a double-stranded DNA or single-stranded DNA recognition cleavage function and a trans single-stranded DNA cleavage function. The CRISPR-Cas nuclease includes one of SpyCas9, FnCas9, FnCas12a, LbCas12, BhCas12b, BsBCasf 2b, LsCas12b, SbCas12b, AaCas12b, AkCas12, AmCas12b, BsCas12b, DiCas12b, TcCas12b, AacCas12b, LwCas13, and Cas14 or one of variants thereof.

In some embodiments, in step (2), a spacer sequence of the guide RNA or derivative thereof is complementary to a sequence of the nucleic acid molecule to be detected.

In some embodiments, in step (2), the OPERATOR reaction buffer comprises 1-5 mM of dNTP, 10-100 mM of Tris-HCl, 5-25 mM of MgCl₂, 0.01-20 mM of ATP, 0.5-10 mM of DTT and 0.5-1.5 mg/ml of bovine serum albumin, and the OPERATOR reaction buffer has a pH between 6.5-8.0.

### BRIEF DESCRIPTION OF THE DRAWINGS

The present disclosure will be further illustrated by way of exemplary embodiments, which will be described in detail through the accompanying drawings. These embodiments are not limiting, wherein:

FIG. 1 is a schematic diagram of an exemplary process for detecting samples according to some embodiments of the present disclosure;

FIG. 2 is a schematic diagram of an exemplary process for detecting ssDNA or RNA samples according to some embodiments of the present disclosure;

FIG. 3 is a schematic diagram of an exemplary process for detecting a DNA sample according to some embodiments of the present disclosure;

FIG. 4 is a graph illustrating the detection results of RNA, dsDNA, and ssDNA molecules according to some embodiments of the present disclosure;

FIG. 5 is a graph illustrating the sensitivity of the detection for RNA samples according to some embodiments of the present disclosure;

FIG. 6 is a histogram illustrating the results of N gene of SARS-CoV-2 detection according to some embodiments of the present disclosure; and

FIG. 7 is a graph comparing the results of the step-by-step and the one-pot method of detection according to some embodiments of the present disclosure.

### DETAILED DESCRIPTION

In order to provide a clearer understanding of the technical solutions of the embodiments described in the present disclosure, a brief introduction to the drawings required in the description of the embodiments is given below. It is evident that the drawings described below are merely some examples or embodiments of the present disclosure, and for those skilled in the art, the present disclosure may be applied to other similar situations without exercising creative labor, unless otherwise indicated or stated in the context, the same reference numerals in the drawings represent the same structures or operations.

It should be understood that, although the terms “first,” “second,” “third,” etc., may be used in the present disclosure to describe various elements, these elements should not be limited by these terms. These terms are used solely to distinguish one element from another. For example, a first product may be referred to as a second product, and similarly, within the scope of exemplary embodiments of the present disclosure, the second product may be referred to as the first product.

Set forth in the present disclosure and the claims, unless explicitly indicated otherwise in the context, words such as “one,” “a,” “an,” and/or “the” do not specifically denote the singular form and may also include the plural form. In general, the terms “comprising” and “including” only suggest the inclusion of steps and elements that have been explicitly identified, and these steps and elements do not constitute an exclusive listing; methods or devices may also include other steps or elements.

Unless otherwise defined, all technical and scientific terms used in the present disclosure have the same meaning as typically understood by those of ordinary skill in the art to which the present disclosure pertains.

In order to overcome the defects of the prior art, the present disclosure provides a one-pot single-stranded DNA cyclization amplification and CRISPR/Cas-mediated nucleic acid molecule detection method, which is an ultrasensitive nucleic acid molecule detection technique using a single-tube, named: OPERATOR. The nucleic acid molecule detection method involves single-stranded DNA probe cyclization, rolling circle replication, and CRISPR/Cas12a cleavage signal amplification detection, specifically involving a new detection method using DNA ligase and displacing amplification enzyme rolling circle amplification in combination with CRISPR-Cas nuclease cleavage, whereby the detection reaction is carried out in parallel with the completion of the amplification. For the detection of RNA samples, OPERATOR technology amplifies RNA molecules directly without a reverse transcription step.

One or more embodiments of the present disclosure provide a one-pot single-stranded DNA cyclization amplification and CRISPR/Cas-mediated nucleic acid molecular detection method, comprising the following steps.

Step (1): extracting a nucleic acid sample from a test sample.

In some embodiments, the nucleic acid molecule to be detected in the nucleic acid sample comprises one or more of single-stranded DNA, double-stranded DNA (dsDNA), or RNA. If the nucleic acid to be detected is double-stranded DNA, the double-stranded DNA is pre-denatured before the reaction.

Step (2): preparing a reaction system mixture, the mixture comprising: a single-stranded DNA probe, a dual fluorescently labelled single-stranded DNA probe, an oligonucleotide primer, a DNA ligase or variants thereof, a strand displacing DNA polymerase or variants thereof, a guide RNA or derivatives thereof, a CRISPR-associated Cas protein or variants thereof, an OPERATOR reaction buffer; wherein the guide RNA or derivatives thereof comprises a sequence identical to a target sequence of the nucleic acid molecule to be detected, the single-stranded DNA probe is specifically complementary to a strand of the nucleic acid molecule to be detected, and a backbone sequence of the single-stranded DNA probe includes a PAM site sequence and a random ligation sequence in addition to the sequence of the complementary target portion or a derivative thereof.

A single-stranded DNA probe (padlock probe) forms a single-stranded circular DNA probe under the action of DNA ligase; the nucleic acid molecule to be detected hybridizes to the single-stranded circular DNA probe and is converted to circular DNA under the action of DNA ligase; the oligonucleotide primer, using the circular DNA as a template, randomly binds to the circular DNA template and extends continuously under the action of strand displacing DNA polymerase to form long-stranded DNA containing repetitive single-stranded DNA probe sequences.

In some embodiments, the single-stranded DNA probe includes a 5′ end, a 3′ end, and a backbone sequence. The 5′ end and the 3′ end of the single-stranded DNA probe are complementary to the sequence of the nucleic acid molecule to be detected, respectively; and the backbone sequence of the single-stranded DNA probe comprises a complementary sequence of the target portion or a derivative thereof, a PAM site sequence, and a random ligation sequence. The random ligation sequences are generally 40-80 bp in length and have a GC content in the range of 30%-70%. A “TTT” PAM site is introduced at the 3′ end of the single-stranded DNA probe sequence for recognition of the target sequence, cyclization, and template amplification, allowing the amplified product to be free of PAM constraints.

In some embodiments, the oligonucleotide primer described in step (2) is a base-modified random primer (6-10 nt in length) or a primer that is consistent with the sequence of the nucleic acid molecule to be detected (10-20 nt). The number of modified bases is 1-10. Random primers are random hexamer primers, and random hexamer primers are random sequence primers containing six bases (NpNpNpNpNpsNs, 6 Ns).

In some embodiments, the sequence of the dual fluorescently labelled single-stranded DNA probe is complementary to the sequence of the nucleic acid molecule to be detected, the 5′ end of the probe is labelled with a fluorescent moiety, and the 3′ end is labelled with a quenching moiety; the fluorescent moiety at the 5′ end of the probe includes one of FAM, HEX, VIC, Cy5, Cy3, ROX, FITC, and Joe, and the fluorescence quenching moiety labelled at the 3′ end includes one of TAMRA, BHQ1, MGB, and BHQ2.

In some embodiments, the DNA ligase in step (2) is a ligase that ligating a double-stranded DNA molecule or a single-stranded DNA nick of an RNA/DNA hybrid double-strand. DNA ligase is capable of specifically ligating phosphodiester bonds of single-stranded DNA complementary to a target. In some embodiments, the DNA ligase may include one of T4 DNA ligase, E. coli DNA ligase, SplintR ligase, and HiFi Taq DNA ligase. In some embodiments, the DNA ligase may comprise a wild-type, modified, codon-optimized, evolved, thermophilic, chimeric, engineered DNA ligases, and/or a mixture of more than one DNA ligase. In some embodiments, the DNA ligase is preferably a T4 DNA ligase.

In some embodiments, the DNA ligase is capable of specifically ligating phosphodiester bonds of ssDNA complementary to the target.

In some embodiments, the strand displacing DNA polymerase described in step (2) may include one of Phi29, Klenow, and Vent. In some embodiments, the strand displacing DNA polymerase is preferably a Phi29 DNA polymerase. The strand displacing DNA polymerase is capable of recognizing a random primer and triggering a strand displacing amplification reaction to produce single-stranded DNA under the guidance of the random primer.

In some embodiments, the strand displacing DNA polymerase may include wild-type, modified, codon-optimized, evolved, thermophilic, chimeric, engineered strand displacing DNA polymerase, and/or a mixture of more than one reverse transcriptase.

In some embodiments, the CRISPR-associated Cas protein is a CRISPR-Cas nuclease having a double-stranded DNA or single-stranded DNA recognition cleavage function and a trans single-stranded DNA cleavage function. In some embodiments, the CRISPR-Cas nuclease includes SpyCas9, FnCas9, FnCas12a, LbCas12, BhCas12b, Bs3Cas12b, LsCas12b, SbCas12b, AaCas12b, AkCas12, AmCas12b, BsCas12b, DiCas12b, TcCas12b, AacCas12b, LwCas13, Cas14, or one of variants thereof. In some embodiments, a CRISPR-Cas nuclease may include a wild-type, modified, codon-optimized, evolved, thermophilic, chimeric, engineered CRISPR-Cas nuclease, and/or a mixture of more than one CRISPR-associated Cas protein. In some embodiments, the CRISPR-Cas nuclease is preferably Cas12a. CRISPR-Cas nuclease-binding guide RNA (crRNA) may be specifically activated by the target nucleic acid sequence thereby possessing a non-specific DNA nuclease activity that enables the cleavage of DNA fluorescent probes.

In some embodiments, in step (2), the spacer sequence of the guide RNA or derivative thereof is complementary to the sequence of the nucleic acid molecule to be detected.

In some embodiments, in step (2), the OPERATOR reaction buffer comprises 1-5 mM of dNTP, 10-100 mM of Tris-HCl, 5-25 mM of MgCl₂, 0.01-20 mM of ATP, 0.5-10 mM of DTT and 0.5-1.5 mg/ml of bovine serum albumin, and the OPERATOR reaction buffer has a pH between 6.5-8.0.

In some embodiments, the OPERATOR reaction buffer may comprise 4 mM of dNTP, 40 mM of Tris-HCl, 10 mM of MgCl₂, 0.5 mM of ATP and 10 mM of DTT, 0.5 mg/ml of bovine serum protein, and the buffer pH is 7.5.

In some embodiments, the OPERATOR reaction buffer may comprise 4 mM of dNTP, 40 of mM Tris-HCl, 10 of mM MgCl₂, 10 mM of DTT, 0.5 mM of ATP, and the buffer pH is 7.8.

In some embodiments, the OPERATOR reaction buffer may comprise 4 mM of dNTP, 50 mM of Tris-HCl, 10 mM of MgCl₂, 10 mM of (NH4)₂SO₄, 4 mM of DTT, 0.5 mM of ATP, and the buffer pH is 7.5.

In some embodiments, the OPERATOR reaction buffer may comprise 4 mM of dNTP, 50 mM of NaCl, 10 mM of Tris-HCl, 10 mM of MgCl₂, 0.5 mM of ATP, and the buffer pH is 7.9.

In some embodiments, the cyclized padlock probe triggers an efficient strand displacing amplification reaction triggered by a random primer, and the random primer is a 6 nt DNA random primer in length with a final concentration of not less than 10 μM. Random primer is capable of triggering highly efficient rolling circle amplification, which is much more efficient than conventional single-primer-triggered amplification.

Step (3): adding the nucleic acid sample to the reaction system mixture for a constant temperature reaction.

In some embodiments, the temperature of the thermostatic reaction is 37° C., and the reaction time is 1 h. In some embodiments, the temperature of the thermostatic reaction is 37° C. and the reaction time is 1.5 h.

Single-stranded DNA probe forms a single-stranded circular DNA probe under the action of DNA ligase; during the thermostatic reaction, the nucleic acid molecule to be detected hybridizes with the single-stranded circular DNA probe and is converted into circular DNA under the action of DNA ligase; oligonucleotide primer takes the cyclic DNA as a template, randomly binds to the circular DNA template and continuously extends under the action of strand displacing DNA polymerase to form a long-stranded DNA containing the sequence of the repetitive single-stranded DNA probe; the long-stranded DNA combines with the dual fluorescently labelled single-stranded DNA probe to form complementary double-stranded DNA; the formed double-stranded DNA is recognized by the guide RNA and the Cas protein complex and cleaves the dual fluorescently labelled single-stranded DNA probe, producing a detectable fluorescent signal.

Step (4): producing a detectable fluorescent signal after the dual fluorescently labelled single-stranded DNA probe is cleaved, reading and recording the fluorescent signal, and obtaining nucleic acid detection results.

One or more embodiments of the present disclosure provide an isothermal nucleic acid detection kit based on a one-pot single-stranded DNA cyclization amplification and CRISPR/Cas-mediated nucleic acid molecule detection method, which allows precise, rapid and highly sensitive detection of specific RNA or DNA molecules at room temperature under isothermal conditions.

In some embodiments, the isothermal nucleic acid detection kit may comprise an enzyme mix, a single-stranded DNA probe, a guide RNA, a dual fluorescently labelled single-stranded DNA probe, an oligonucleotide primer, and an OPERATOR reaction buffer.

In some embodiments, the enzyme mixture may comprise a CRISPR-Cas nuclease, a DNA ligase, and a strand displacing DNA polymerase.

In some embodiments, the CRISPR-Cas nuclease is preferably FnCas12a; the DNA ligase is preferably T4 DNA ligase; and the strand displacing DNA polymerase is preferably Phi29 DNA polymerase.

In some embodiments, the padlock probe (single-stranded DNA probe) consists of a sequence complementary to the target sequence with a loop backbone sequence, and a “TTT” PAM site is introduced at the 3′ end of the padlock probe sequence.

In some embodiments, the dual fluorescently labelled single-stranded DNA probe is single-stranded DNA labelled with a fluorescent moiety at the 5′ end and a fluorescence quenching moiety at the 3′ end.

In some embodiments, the oligonucleotide primer may be a 10 μM-100 μM random hexamer primer.

In some embodiments, the OPERATOR reaction buffer may comprise 1-5 mM of dNTP, 10-100 mM of Tris-HCl, 5-25 mM of MgCl₂, 0.01-20 mM of ATP and 0.5-10mM of DTT, 0.1-1.5 mg/ml of bovine serum protein, and the buffer pH is between 6.5-8.0.

In some embodiments, the isothermal nucleic acid detection kit may comprise random primers 6Ns (10 μM-100 μM), FAM-labelled fluorescent probes 1-4 nM, enzyme mixtures (T4 DNA ligase, 5U-200U; Phi29 DNA polymerase, 5 U-20 U; Cas12a protein, 0.1 μg-5 μg) and as described in the preceding embodiments OPERATOR reaction buffer.

In some embodiments, the target DNA, the guide RNA, and the Cas12a protein may form a complex, and the complex cleaves other single-stranded DNA molecules in the system.

One or more embodiments of the present disclosure provide one-pot single-stranded DNA circular amplification and CRISPR/Cas-mediated nucleic acid molecule detection method and kits for detecting nucleic acid molecules of bacteria, fungi, viruses, human or other plant and animal tissues.

One or more embodiments of the present disclosure provide a reaction system, the system having: a single-stranded DNA probe, a dual fluorescently labelled single-stranded DNA probe, an oligonucleotide primer, a DNA ligase and variants thereof, a strand displacing DNA polymerase and variants thereof, clustered of regularly interspaced short palindromic repeat (CRISPR) RNAs (crRNAs) or derivatives thereof, a CRISPR-related (Cas) protein or variants thereof, and OPERATOR reaction buffer. The crRNA or a derivative thereof comprises a sequence that is identical to a target sequence of the nucleic acid molecule to be detected.

The embodiments of the present disclosure provide a nucleic acid molecule detection method and kit that may rapidly accomplish the detection of a DNA or RNA molecule at room temperature under isothermal conditions. Firstly nucleic acid extraction is performed to obtain RNA, single-stranded DNA, or double-stranded DNA of the sample to be detected; and then a ligase, an amplifying enzyme, a combinatorial enzyme of CRISPR-related proteins, a single-stranded DNA probe, and a nucleic acid fluorescent probe are used to react with the nucleic acid to be detected under isothermal conditions, and finally the fluorescent signal is detected to determine whether the target nucleic acid exists in the sample to be detected.

Embodiments of the present disclosure have at least the following beneficial effects:
(1) High sensitivity: realizing the detection of nucleic acid molecules at the single-copy level;
(2) Versatility: realizing the detection of DNA (single-stranded DNA and double-stranded DNA) or RNA;
(3) Multi-channel: realizing multi-channel detection and detect multiple samples at one time;
(4) Rapid: completing the test as short as 30 min;
(5) Convenient: realizing the isothermal reaction of single buffer in a single tube, which is convenient to operate and with easy steps;
(6) Low false positives: although the reaction system provided in the embodiments of the present disclosure contains an amplification step, the detection and amplification products are RNA and DNA, respectively. And the single-stranded DNA probe is cyclized and thus triggers the amplification reaction only when the RNA target is present, which overcomes the easily contaminated characteristics of LAMP, fluorescence quantitative PCR, and so on. At the same time, the method is a closed-tube reaction, which is physically isolated and minimizes the possibility of contamination;
(7) Ambient isothermal detection: the three engineered enzymes and chemical components work together to create an environment that maximally mimics nucleic acid amplification in vivo, and each engineered enzyme performs its own role, working at its own optimal reaction temperature, so the working efficiency is the highest;
(8) One-step method: a buffer is constructed in which the three reactions are compatible with each other, and the single-stranded DNA probe cyclization, amplification, and Cas 12a cleavage detection reactions are placed in the same reaction tube, enabling the detection of samples by one-time addition of samples, thus making the operation simpler.

The experimental techniques in the following examples, unless otherwise specified, are conventional techniques. The test materials used in the following examples, unless otherwise specified, are obtained from standard biochemical reagent companies. Quantitative detections in the following examples are performed with three replicate experiments, and the results are averaged.

### EXAMPLES

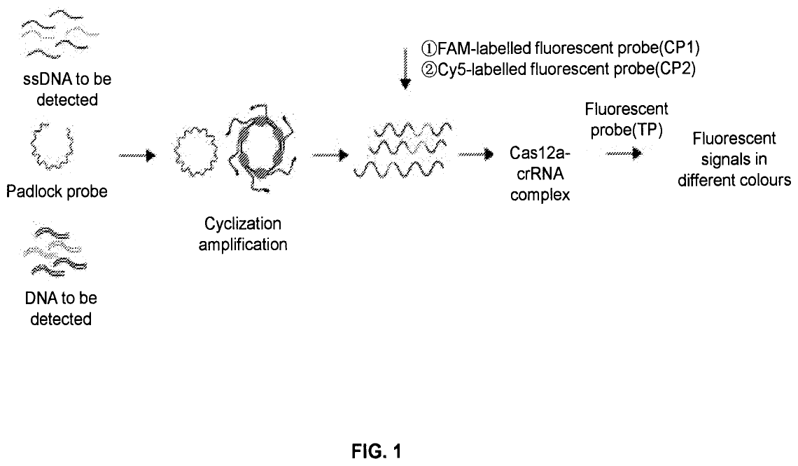

Examples of the present disclosure provide a one-pot for single-stranded DNA cyclization amplification and CRISPR/Cas-mediated nucleic acid molecule detection method, with a process as shown in FIG. 1.

The sequences of primers, probes, etc., used in the Examples are shown in Table 1:

TABLE 1
Sequence list of primers and probes

| | | SEQ.ID. | Amplification |
|---|---|---|---|
| Primer name | Primer sequence (5′-3′) | NO | length |
| Target1 | GGAAGAGACAGGTACGTTAATAGTT | 1 | 210 |
| | AATAGCGTACTTCTTTTTCTTGCTTTC | | |
| | GTGGTATTCTTGCTAGTTACACTAGC | | |
| | CATCCTTACTGCGCTTCGATTGTGTG | | |
| | CGTACTGCTGCAATATTGTTAACGTG | | |
| | AGTCTTGTAAAACCTTCTTTTTACGTT | | |
| | TACTCTCGTGTTAAAAATCTGAATTC | | |
| | TTCTAGAGTTCCTGATCTTCTGGTCT | | |
| | A | | |
| Target2 | TATTGTTAACGTGAGTCTTGTAAAAC | 5 | 58 |
| | CTTCTTTTTACGTTTACTCTCGTGTTA | | |
| | AAAAT | | |
| Target3 | GGAAGAGACAGGUACGUUAAUAGU | 6 | 210 |
| | UAAUAGCGUACUUCUUUUUCUUGC | | |
| | UUUCGUGGUAUUCUUGCUAGUUAC | | |
| | ACUAGCCAUCCUUACUGCGCUUCG | | |
| | AUUGUGUGCGUACUGCUGCAAUAU | | |
| | UGUUAACGUGAGUCUUGUAAAACCU | | |
| | UCUUUUUACGUUUACUCUCGUGUU | | |
| | AAAAAUCUGAAUUCUUCUAGAGUUC | | |
| | CUGAUCUUCUGGUCUA | | |
| PL target 1, | AAGGTTTTACACTTTCCGTCTTTATA | 4 | 60 |
| 2, 3 | GTCTGTCGTATTAATTTCTCTTTAACG | | |
| | TAAAAAG | | |
| crRNA-F | GAAATTAATACGACTCACTATAGGG | 3 | 25 |
| crRNA-target-R | TGTAAAACCTTCTTTTTACGTTATCTA | 2 | 67 |
| | CAACAGTAGAAATTACCCTATAGTGA | | |
| | GTCGTATTAATTTC | | |
| PL-N | TGCCAGCCATTCTTTCCGTCTTTATA | 8 | 60 |
| | GTCTGTCGTA | | |
| | TTAATTTCTCTTTATCACCGCCAT | | |
| crRNA-N-R | ATCACCGCCATTGCCAGCCATTATCT | 9 | 67 |
| | ACAACAGTAGAAATTACCCTATAGTG | | |
| | AGTCGTATTAATTTC | | |
| TP | FAM-TTATTATT-BHQ1 | | 8 |
| CP | FAM- | 10 | 28 |
| | TTTAACGTAAAAAGAAGGTTTTACAC | | |
| | TT-BHQ1 | | |
| 6Ns | NpNpNpNpNpsNs | | 5 |

### Example 1

### Detection of Double-Stranded DNA (dsDNA) Target

The dsDNA (Target 1) was selected as the target sequence and the Target 1 sequence is shown in SEQ ID NO.1:

(SEQ ID NO. 1)
GGAAGAGACAGGTACGTTAATAGTTAATAGCGTACTTCTTTTTCTTGCT
TTCGTGGTATTCTTGCTAGTTACACTAGCCATCCTTACTGCGCTTCGAT
TGTGTGCGTACTGCTGCAATATTGTTAACGTGAGTCTTGTAAAACCTTC
TTTTTACGTTTACTCTCGTGTTAAAAATCTGAATTCTTCTAGAGTTCCT
GATCTTCTGGTCTA.

Preparation of guide RNA: the reverse complementary length primer crRNA-target-R containing the T7 sequence was synthesized as shown in SEQ ID NO.2:

(SEQ ID NO. 2)
TGTAAAACCTTCTTTTTACGTTATCTACAACAGTAGAAATTACCCTATA
GTGAGTCGTATTAATTTC.

The forward primer crRNA-F for T7 is shown in SEQ ID NO. 3:

(SEQ ID NO. 3)
GAAATTAATACGACTCACTATAGGG.

Incomplete DNA double strands were obtained by double primer annealing and stored at −20° C. or −80° C. for backup.

The single-stranded DNA probe sequence for Target 1 is shown in SEQ ID NO.4:

(SEQ ID NO. 4)
AAGGTTTTACACTTTCCGTCTTTATAGTCTGTCGTATTAATTTCTCTTT
AACGTAAAAAG.

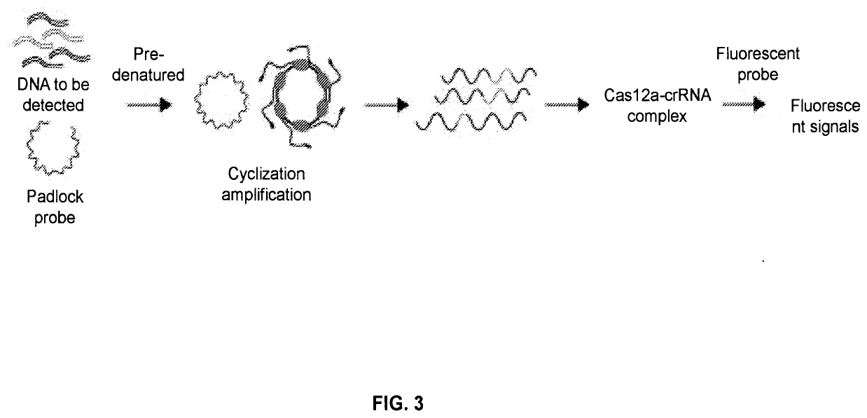

The amplification and detection reaction were shown in FIG. 3. Firstly, the 100 nM single-stranded DNA probe was annealed with the dsDNA to be detected at high temperature (85-95° C.) for 5 min, and then added into a reaction system after natural cooling. The reaction system consisted of the following: buffer (1×) (4 mM dNTP, 40 mM Tris-HCl, 10 mM MgCl₂, 0.5 mM ATP and 10 mM DTT, 0.5 mg/ml bovine serum protein, and a buffer pH of 7.5), 10 μM random primer 6 Ns (NpNpNpNpNpsNs), 100 nM guide RNA, 200 nM FAM dual fluorescently labelled probe, enzyme mixture (5 U T4 DNA ligase, 10 U Phi29 DNA polymerase, 250 nM Cas12a protein).

Fluorescence detection: after the reaction was mixed, the temperature was set to 37° C. in 7900HT Fast Real-Time RCR system, the fluorescence detection probe was FAM, and the sequence of trans-probe (TP) is: TTATTATT; the sequence of cis-probe (CP) is as shown in SEQ ID NO.10: TTTAACGTAAAAAGAAGGTTTTACACTT (SEQ ID NO.10); the fluorescence signal acquisition time interval was 1 min, and the detection time was 1 h.

Specific reaction process: the above single-stranded DNA probe was annealed with dsDNA to be detected at high temperature (85-95° C.) for 5 min, then added into the integrated system of amplification and reaction after natural cooling, and reacted at a constant temperature of 37° C. for 1 h, and fluorescence signal was detected synchronously using 7900HT Fast Real-Time RCR system with a fluorescence signal acquisition time interval of 1 min and detection time of 1 h.

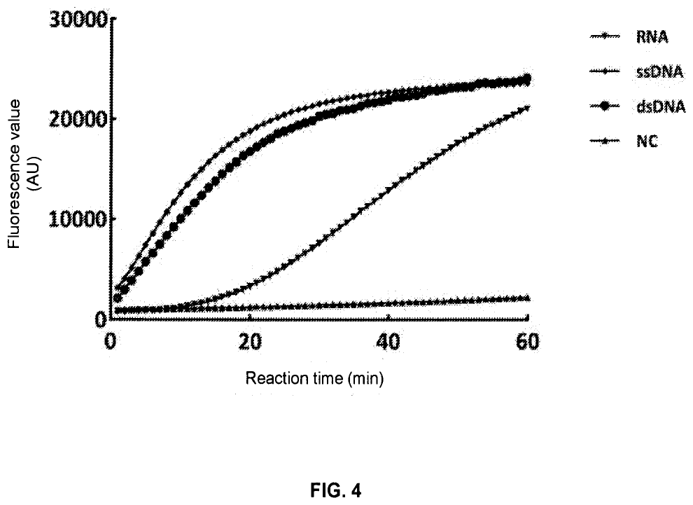

The results of the detection are as shown in FIG. 4, indicating that the nucleic acid molecular detection provided in the present disclosure can be used for the detection of double-stranded DNA.

### Example 2

### Detection of Single-Stranded DNA (ssDNA) Target

The ssDNA (Target 2) was selected as the target sequence and the Target 2 sequence is shown in SEQ ID NO.5:

(SEQ ID NO. 5)
TATTGTTAACGTGAGTCTTGTAAAACCTTCTTTTTACGTTTACTCTCGT
GTTAAAAAT

Preparation of target single-stranded DNA: primer (Target 2) as shown in SEQ ID NO.5 was synthesized and dissolved in water and diluted to 10 uM.

Preparation of guide RNA: the reverse complementary length primer crRNA-target-R containing the T7 sequence was synthesized as shown in SEQ ID

NO.2:
(SEQ ID NO. 2)
TGTAAAACCTTCTTTTTACGTTATCTACAACAGTAGAAATTACCCTATA
GTGAGTCGTATTAATTTC.

The forward primer crRNA-F for T7 is shown in SEQ ID NO.3:

(SEQ ID NO. 3)
GAAATTAATACGACTCACTATAGGG.

Incomplete DNA double strands were obtained by double primer annealing and stored at −20° C. or −80° C. for backup.

The single-stranded DNA probe sequence for Target 2 is shown in SEQ ID NO.4:

(SEQ ID NO. 4)
AAGGTTTTACACTTTCCGTCTTTATAGTCTGTCGTATTAATTTCTCTTT
AACGTAAAAAG

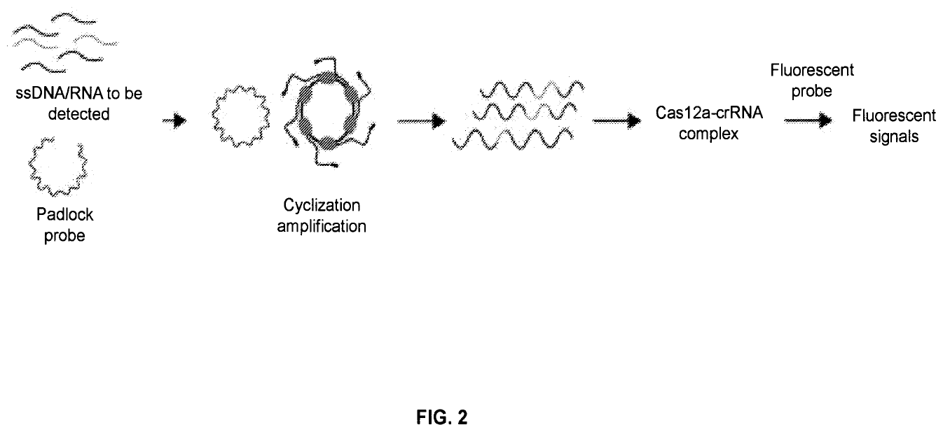

As shown in FIG. 2, the amplification and detection reaction: ssDNA to be detected was added to the reaction system, which consisted of: buffer (1×) (4 mM dNTP, 40 mM Tris-HCl, 10 mM MgCl₂, 0.5 mM ATP and 10 mM DTT, 0.5 mg/ml bovine serum proteins, and a buffer pH of 7.5), 100 nM single-stranded DNA probes, 10 μM random primer 6 Ns (NpNpNpNpNpsNs), 100 nM guide RNA, 200 nM FAM dual fluorescently labelled probe, enzyme mixture (5 U T4 DNA ligase, 10 U Phi29 DNA polymerase, 250 nM Cas12a protein).

Fluorescence detection: after the reaction was mixed, the temperature was set to 37° C. in 7900HT Fast Real-Time RCR system, and the fluorescence detection probe was FAM, as shown in Table 1. The fluorescence signal acquisition time interval was 1 min, and the detection time was 1 h.

The result of the detection is shown in FIG. 4, indicating that the nucleic acid molecular detection provided in the present disclosure can be used for the detection of single-stranded DNA.

### Example 3

### Detection of RNA Target

RNA (Target 3) was selected as the target sequence and the Target 3 sequence is shown in SEQ ID NO.6:

(SEQ ID NO. 6)
GGAAGAGACAGGUACGUUAAUAGUUAAUAGCGUACUUCUUUUUCUUGCU
UUCGUGUGUAUUCUUGCUAGUUUACACUAGCCAUCCUACUGCUUCGAUU
GUGCGUACUGCUGCAAUUAUUUUUUAACGUGAGUCUUUUAAAACCUUCU
UUUUUUUUUUUUUUUUUUUUUUUUUUUUUUUUUUUUUUUUUUUUUUUUU
UUUUUUUUUUUUUUUUCUCUUUUUUUUUUUUUUUUUUUUUUUUUUUUUU
UUUUUUUUUUGGUCUCUUUUUUA.

Preparation of guide RNA: the reverse complementary long primer crRNA-target-R containing the T7 sequence was synthesized as shown in SEQ ID NO.2, TGTAAAACCTTCTTTTTACGTTATCTACAACAGTAGAAATTACCCTATAGTGAGTCG TATTAATTTC (SEQ ID NO.2); the forward primer crRNA-F for T7 is shown in SEQ ID NO.3: GAAATTAATACGACTCACTATAGGG (SEQ ID NO.3).

Incomplete DNA double strands were obtained by double primer annealing and stored at −20° C. or −80° C. for backup.

The single-stranded DNA probe sequence for Target 3 is shown in SEQ ID NO.4:

(SEQ ID NO. 4)
AAGGTTTTACACTTTCCGTCTTTATAGTCTGTCGTATTAATTTCTCTTT
AACGTAAAAAG.

Amplification and detection reaction: the RNA to be detected was added to the reaction system, which consisted of: buffer (1×) (4 mM dNTP, 40 mM Tris-HCl, 10 mM MgCl₂, 0.5 mM ATP, 10 mM DTT, 0.5 mg/ml bovine serum proteins, and a buffer pH of 7.5), 100 nM single-stranded DNA probes, 10 μM random primers 6 Ns (NpNpNpNpNpsNs), 100 nM guide RNA, 200 nM FAM dual fluorescently labelled probe, enzyme mixture (5U T4 DNA ligase, 10U Phi29 DNA polymerase, 250 nM Cas12a protein).

Fluorescence detection: after reaction was mixed, the temperature was set to 37° C. in 7900 HT Fast Real-Time RCR system, and the fluorescence detection probe was FAM, as shown in Table 1. The fluorescence signal acquisition time interval was 1 min, and the detection time was 1 h.

Specific reaction process: the above single-stranded DNA probe and RNA to be detected were added into the integrated system of amplification and reaction, and the reaction was carried out at a constant temperature of 37° C. for 1 h, and fluorescence signal was detected synchronously using 7900HT Fast Real-Time RCR system with a fluorescence signal acquisition time interval of 1 min and detection time of 1 h.

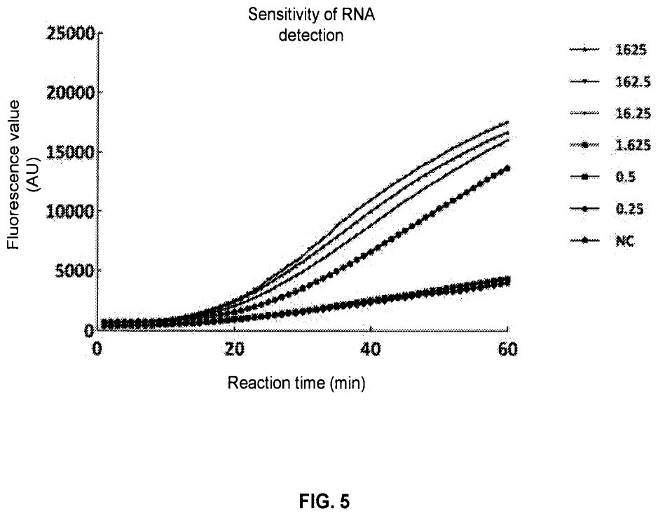

The detection results are shown in FIG. 4, indicating that the nucleic acid molecular detection provided in the present disclosure can detect single-stranded RNA. FIG. 5 is a graph of the sensitivity of detection of RNA samples according to some embodiments shown in the present disclosure. Single-stranded RNA molecules as low as 1.625 copies/μl can be detected by applying the nucleic acid molecule detection provided in the present disclosure, as shown in FIG. 5.

### Example 4

### Detection of SARS-COV-2

SARS-COV-2 is an RNA virus, and total RNA was extracted from the nasopharyngeal samples, and the total RNA extracted was used as the RNA to be detected.

The sequence of N gene of SARS-COV-2 was selected as the target sequence, and the sequence of the conserved region of N gene of SARS-COV-2 is shown in SEQ ID NO.7: AAUGGCUGGGCAAUGGGGGGUGAU (SEQ ID NO.7).

The padlock probe (PL-N) sequence of N gene of SARS-COV-2 was selected as shown in SEQ ID NO.8:

(SEQ ID NO. 8)
TGCCAGCCATTCTTTCCGTCTTTATAGTCTGTCGTATTAATTTCTTTAT
CACCGCCAT.

Preparation of guide RNA: the reverse complementary long primer crRNA-N-R containing the sequence of T7 was synthesized as shown in SEQ ID NO.9: ATCACCGCCATTGCCAGCCATTATCTACAACAGTAGAGAAATTACCCTATAGTGAG TCGTA TTAATTTC (SEQ ID NO.9); T7's forward primer crRNA-F is shown in SEQ ID NO.3: GAAATTAATACGACTCACTATAGGG (SEQ ID NO.3).

Incomplete DNA double strands were obtained by double primer annealing and stored at −20° C. or −80° C. for backup.

Amplification and detection reaction: RNA to be examined was added to the reaction system, which consisted of: buffer (1-5 mM dNTP, 10-100 mM Tris-HCl, 5-25mM MgCl₂, 0.01-20 mM ATP, 0.5-10 mM DTT, 0.1-1.5 mg/ml bovine serum proteins, and a buffer pH between 6.5-8.0), random primers 10 μM-100 μM 6 Ns; 100 nM-400 nM single-stranded DNA probe, 100 nM-400 nM guide RNA, 1-4 nM FAM dual fluorescently labelled probe, enzyme mixtures (5U-200U T4 DNA ligase, 5U-20U Phi29 DNA polymerase, 0.1 ug-5ug Cas12a protein).

Fluorescence detection: after reaction was mixed, the temperature was set to 37° C. in 7900 HT Fast Real-Time RCR system, and the fluorescence detection probe was FAM, as shown in Table 1, and the fluorescence signal acquisition time interval was 1 min, and the detection time was 1 h.

Specific reaction process: the above single-stranded DNA probe and RNA to be detected were added into the integrated system of amplification and reaction, and the reaction was carried out at a constant temperature of 37° C. for 1 h, and synchronized with the fluorescence signal detection by using the 7900 HT Fast Real-Time RCR system with a fluorescence signal acquisition time interval was 1 min, and the detection time was 1 h.

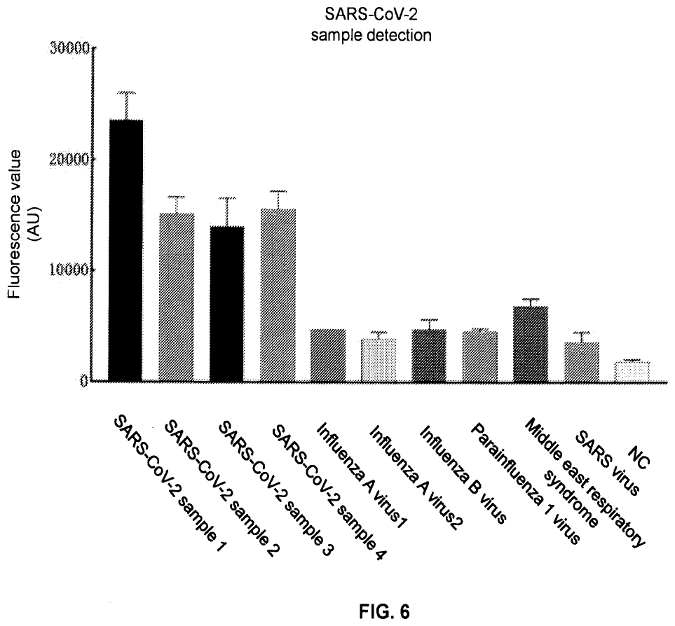

The results of the detection were shown in FIG. 6, indicating that the nucleic acid molecular detection provided in the present disclosure can be used for SARS-COV-2 detection.

### Comparative Example 1

The steps of the one-pot single-stranded DNA cyclization amplification and CRISPR/Cas-mediated nucleic acid molecule detection method in the comparative example 1 are essentially the same as those of the examples, differing only in the following: the Buffer 1 (B1) used for ssDNA cyclization in the substeps (40 mM Tris-HCl, 10 mM MgCl₂, 10 mM DTT, 0.5 mM ATP, and a buffer pH of 7.8); Phi29 amplification Buffer 2 (B2) (50 mM Tris-HCl, 10 mM MgCl₂, 10 mM (NH4)2SO4, 4 mM DTT, and a buffer pH of 7.5) and CRISPR/Cas-mediated nucleic acid detection Buffer 3 (B3) (50mM NaCl, 10 mM Tris-HCl, 10 mM MgCl₂, 100 μg/ml bovine serum proteins, and a buffer PH of 7.9) were optimized as one-pot reaction Buffer (B) (4 mM dNTP, 40 mM Tris-HCl, 10 mM MgCl₂, 0.5 mM ATP, 10 mM DTT, 0.5 mg/ml bovine serum proteins, and a buffer PH of 7.5), in order to verify the effect after optimization, the above RNA (Target 3) was chosen as the target sequence for comparison of B1, B2, B3, and one-pot reaction B, respectively, and the Target 3 sequence is shown in SEQ ID NO.6:

(SEQ ID NO. 6)
GGAAGAGACAGGUACGUUAAUAGUUAAUAGCGUACUUCUUUUUUCUUGC
UUUCGUGUGUAUUCUUGCUAGUUUACACUAGCCAUCCUUACUGCUUCUG
AUGUGCGUACUGCUGCAAUUAUUUUUUUAACGUGAGUCUUUUUAAAACA
CCUUUUUUUUUUUUUUUUUUUUUUUUUUUUUUUUUUUUUUUUUUUUUUU
UUUUUUUUUUUUUUUUUUCUCUUUUUUUUUUUUUUUUUUUUUUUUUUUU
UUUUUUUUUUUUUGGUCUUUUA.

Preparation of guide RNA: the reverse complementary long primer crRNA-target-R containing the T7 sequence was synthesized as shown in SEQ ID NO.2: TGTAAAACCTTCTTTTTACGTTATCTACAACAGTAGAAATTACCCTATAGTGAGTCG TATTAATTTC (SEQ ID NO.2); the forward primer crRNA-F for T7 is shown in SEQ ID NO.3: GAAATTAATACGACTCACTATAGGG (SEQ ID NO.3).

Incomplete DNA double strands were obtained by double primer annealing and stored at −20° C. or −80° C. for backup.

The sequence of the single-stranded DNA probe for Target 3 is shown in SEQ ID NO.4:

(SEQ ID NO. 4)
AAGGTTTTACACTTTCCGTCTTTATAGTCTGTCGTATTAATTTCTCTTT
AACGTAAAAAG

One-pot detection reaction: the RNA to be detected was added to the reaction system, and the one-pot B1 reaction system consisted of: buffer (1×) (4 mM dNTP, 40 mM Tris-HCl, 10 mM MgCl₂, 10 mM DTT, 0.5 mM ATP, and a buffer pH of 7.8). The one-pot method B2 reaction system consisted of: buffer (1×) (4 mM dNTP, 50 mM Tris-HCl, 10 mM MgCl₂, 10 mM (NH4)2SO4, 4 mM DTT, 0.5 mM ATP, and a buffer pH of 7.5). The one-pot method B3 reaction system consisted of: buffer (1×) (4 mM dNTP, 50 mM NaCl, 10 mM Tris-HCl, 10 mM MgCl₂, 0.5 mM ATP, and a buffer pH of 7.9). The optimized reaction system Buffer (B) for the one-pot method consisted of: buffer (1×) (4 mM dNTP, 40 mM Tris-HCl, 10 mM MgCl₂, 0.5 mM ATP, 10 mM DTT, and 0.5 mg/ml bovine serum protein, and a buffer pH of 7.5). In addition, each reaction system further included: random primers 10 UM of 6 Ns (NpNpNpNpNpsNs), 200 nM of FAM-labelled fluorescent probes, and enzyme mixtures (5 U T4 DNA ligase, 10 U Phi29 DNA polymerase, 250 nM Cas12a protein). After each reaction system was mixed, the temperature was set at 37° C. in a 7900 HT Fast Real-Time RCR system, the fluorescence detection probe was FAM, the fluorescence signal acquisition interval was 1 min, and the detection time was 1.5 h.

Specific reaction process: the above single-stranded DNA probe and RNA to be detected were added into the integrated system of amplification and reaction, and the reaction was carried out at a constant temperature of 37° C. for 1.5 h, and synchronized with a 7900 HT Fast Real-Time RCR system for fluorescence signal detection, the fluorescence signal acquisition time interval is 1 min, detection time is 1.5 h.

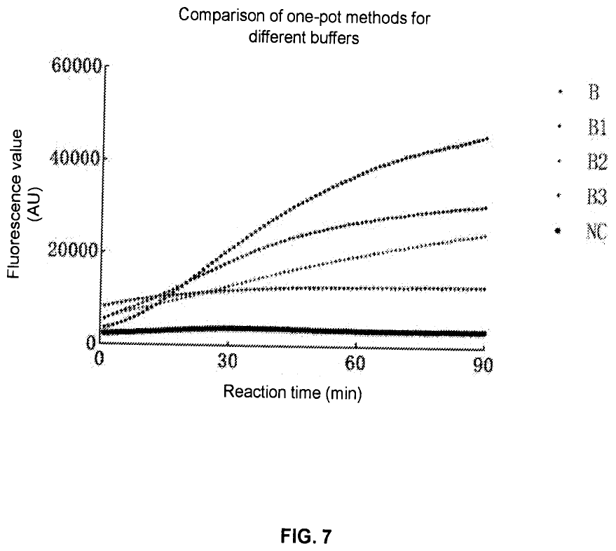

The comparative analysis results are shown in FIG. 7. The results indicates that the one-pot method with different buffers can detect RNA, and the optimized buffer for one-pot method is better than B1, B2 and B3. The detection time can be shortened to 30 min using the one-pot method.

The basic concepts have been described above, and it is apparent to those skilled in the art that the foregoing detailed disclosure is intended as an example only and does not constitute a limitation of the present disclosure. Although not expressly stated herein, those skilled in the art may make various modifications, improvements, and amendments to the present disclosure. Such modifications, improvements, and amendments are suggested in the present disclosure, so such modifications, improvements, and amendments remain within the spirit and scope of the exemplary embodiments of the present disclosure.

At the same time, specific terms are employed to describe the embodiments of the present disclosure. Terms e.g., “an embodiment,” “one embodiment,” and/or “some embodiments” are intended to refer to one or more features, structures, or features associated with at least one embodiment of the present disclosure. Thus, it should be emphasized and noted that the terms “an embodiment,” “one embodiment,” or “an alternative embodiment,” mentioned at different locations in the present disclosure two or more times, do not necessarily refer to a same embodiment. Additionally, certain features, structures, or features of one or more embodiments of the present disclosure may be appropriately combined.

Some embodiments use numbers to describe the number of components, and attributes, and it should be understood that such numbers used in the description of the embodiments are modified in some examples by the modifiers “about”, “approximately”, or “generally”. Unless otherwise stated, “about”, “approximately” or “generally” indicates that a variation of +20% is permitted. Accordingly, in some embodiments, the numerical parameters used in the present disclosure and claims are approximations, which may change depending on the desired features of the individual embodiment. In some embodiments, the numeric parameters should be considered with the specified significant figures and be rounded to a general number of decimal places. Although the numerical domains and parameters configured to confirm the breadth of their ranges in some embodiments of the present disclosure are approximations, in specific embodiments such values are set as precisely as possible within the feasible range.

With respect to each patent, patent application, patent application disclosure, and other material, e.g., articles, books, manuals, publications, documents, etc., cited in the present disclosure, the entire contents thereof are hereby incorporated herein by reference. Application history documents that are inconsistent with or conflict with the contents of the present disclosure are excluded, as are documents (currently or hereafter appended to the present disclosure) that limit the broadest scope of the claims of the present disclosure. It should be noted that in the event of any inconsistency or conflict between the descriptions, definitions, and/or use of terminology in the materials appended to the present disclosure and those described in the present disclosure, the descriptions, definitions, and/or use of terminology in the present disclosure shall prevail.

In closing, it should be understood that the embodiments described in the present disclosure are intended only to illustrate the principles of the embodiments of the present disclosure. Other deformations may also fall within the scope of the present disclosure. Thus, by way of example and not limitation, alternative configurations of embodiments of the present disclosure may be considered consistent with the teachings of the present disclosure. Accordingly, the embodiments of the present disclosure are not limited to the embodiments expressly presented and described herein.

What is claimed is:

1. A one-pot single-stranded DNA cyclization amplification and CRISPR/Cas-mediated nucleic acid molecule detection method, comprising:
step (1): extracting a nucleic acid sample from a test sample;
step (2): preparing a reaction system mixture, the reaction system mixture comprising: a single-stranded DNA probe, a dual fluorescently labelled single-stranded DNA probe, an oligonucleotide primer, a DNA ligase or variants thereof, a strand displacing DNA polymerase or variants thereof, a guide RNA or derivatives thereof, a CRISPR-associated Cas protein or variants thereof, an OPERATOR reaction buffer; wherein the guide RNA or derivatives thereof comprises a sequence identical to a sequence of a nucleic acid molecule to be detected in the nucleic acid sample, the single-stranded DNA probe is specifically complementary to a strand of the nucleic acid molecule to be detected, and a backbone sequence of the single-stranded DNA probe includes a PAM site sequence and a random ligation sequence in addition to a sequence of a complementary target portion or a derivative thereof;
step (3): adding the nucleic acid sample to the reaction system mixture for a constant temperature reaction; and
step (4): producing a detectable fluorescent signal after the dual fluorescently labelled single-stranded DNA probe is cleaved, reading and recording the fluorescent signal, and obtaining a nucleic acid detection result.

2. The method of claim 1, wherein in step (1), the nucleic acid molecule to be detected in the nucleic acid sample comprises one or more of single-stranded DNA, double-stranded DNA, or single-stranded RNA.

3. The method of claim 1, wherein in step (2), the oligonucleotide primer is a base-modified random primer or a primer consistent with the sequence of the nucleic acid molecule to be detected.

4. The method of claim 1, wherein in step (2), the sequence of the dual fluorescently labelled single-stranded DNA probe is complementary to the sequence of the nucleic acid molecule to be detected, and the 5′ end and the 3′ end of the dual fluorescently labelled single-stranded DNA probe are labelled with fluorescent moieties, respectively; and the fluorescent moiety at the 5′ end comprises one of FAM, HEX, VIC, Cy5, Cy3, ROX, FITC, and Joe, and the fluorescence quenching moiety labelled at the 3′ end comprises one of TAMRA, BHQ1, MGB, and BHQ2.

5. The method of claim 1, wherein in step (2), the DNA ligase is a ligase that joins single-stranded DNA nicks of a double-stranded DNA molecule or an RNA and DNA hybrid double strand; the DNA ligase comprises one of T4 DNA ligase, E. coli DNA ligase, SplintR ligase, and HiFi Taq DNA ligase; the strand displacing DNA polymerase includes one of Phi29, Klenow, and Vent.

6. The method of claim 1, wherein in step (2), the CRISPR-associated Cas protein is a CRISPR-Cas nuclease having a double-stranded DNA or single-stranded DNA recognition cleavage function and a trans single-stranded DNA cleavage function; and the CRISPR-Cas nuclease includes one of SpyCas9, FnCas9, FnCas12a, LbCas12, BhCas12b, Bs3Cas12b, LsCas12b, SbCas12b, AaCas12b, AkCas12, AmCas12b, BsCas12b, DiCas12b, TcCas12b, AacCas12b, LwCas13, and Cas14 or one of variants thereof.

7. The method of claim 1, wherein in step (2), a spacer sequence of the guide RNA or derivatives thereof is complementary to a sequence of the nucleic acid molecule to be detected.

8. The method of claim 1, wherein in step (2), the OPERATOR reaction buffer comprises 1-5 mM of dNTP, 10-100 mM of Tris-HCl, 5-25 mM of MgCl₂, 0.01-20 mM of ATP, 0.5-10 mM of DTT and 0.5-1.5 mg/ml of bovine serum albumin, and the OPERATOR reaction buffer has a pH between 6.5-8.0.


SEQUENCE LISTING (10 sequences)

SEQ ID NO: 1 - DNA, 210 bp, synthetic construct
  source 1..210: mol_type = other DNA
ggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtcta

SEQ ID NO: 2 - DNA, 67 bp, synthetic construct
  source 1..67: mol_type = other DNA
tgtaaaaccttctttttacgttatctacaacagtagaaattaccctatagtgagtcgtattaatttc

SEQ ID NO: 3 - DNA, 25 bp, synthetic construct
  source 1..25: mol_type = other DNA
gaaattaatacgactcactataggg

SEQ ID NO: 4 - DNA, 60 bp, synthetic construct
  source 1..60: mol_type = other DNA
aaggttttacactttccgtctttatagtctgtcgtattaatttctctttaacgtaaaaag

SEQ ID NO: 5 - DNA, 58 bp, synthetic construct
  source 1..58: mol_type = other DNA
tattgttaacgtgagtcttgtaaaaccttctttttacgtttactctcgtgttaaaaat

SEQ ID NO: 6 - RNA, 210 nt, synthetic construct
  source 1..210: mol_type = other RNA
ggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtcta

SEQ ID NO: 7 - RNA, 22 nt, synthetic construct
  source 1..22: mol_type = other RNA
aatggctggcaatggcggtgat

SEQ ID NO: 8 - DNA, 60 bp, synthetic construct
  source 1..60: mol_type = other DNA
tgccagccattctttccgtctttatagtctgtcgtattaatttctctttatcaccgccat

SEQ ID NO: 9 - DNA, 67 bp, synthetic construct
  source 1..67: mol_type = other DNA
atcaccgccattgccagccattatctacaacagtagaaattaccctatagtgagtcgtattaatttc

SEQ ID NO: 10 - DNA, 28 bp, synthetic construct
  source 1..28: mol_type = other DNA
tttaacgtaaaaagaaggttttacactt